(19) United States
(12) Patent Application Publication
(10) Pub. No.: US 2026/0007844 A1
(43) Pub. Date: Jan. 8, 2026

(54) VENTILATOR FOR SUPPLYING RESPIRATORY GAS

(71) Applicants: RHEINISCH-WESTFÄLISCHE TECHNISCHE HOCHSCHULE (RWTH) AACHEN, ABGERKÜRZT RWTH AACHEN, KÖRPERSCHAFT DES, Aachen (DE); LÖWENSTEIN MEDICAL TECHNOLOGY SA, Luxemburg (LU)

(72) Inventors: Mateusz BUGLOWSKI, Aachen (DE); Valerie PFANNSCHMIDT, Aachen (DE); André STOLLENWERK, Aachen (DE); Mark SCHOBERER, Aachen (DE); Thi Bich Huong NGUYEN, Aachen (DE); Oliver BRAUN, Bad Ems (DE); Sabine BECKER, Aachen (DE)

(21) Appl. No.: 18/992,470

(22) PCT Filed: Jul. 5, 2024
(86) PCT No.: PCT/EP2023/068525

§ 371 Date: Mar. 11, 2025

(30) Foreign Application Priority Data
Jul. 8, 2022 (DE) 102022117141.8

(51) Int. Cl. A61M 16/00; A61M 16/08

(52) U.S. Cl. CPC A61M 16/024; A61M 16/0003; A61M 16/0875; A61M 2202/0007; A61M 2202/02; A61M 2205/3303; A61M 2205/3327; A61M 2205/3331; A61M 2205/502; A61M 2230/005; A61M 2230/432; A61M 2240/00

(57) ABSTRACT

The invention relates to a ventilator (100) and a control device (120) for controlling a respiratory gas source of a ventilator, in particular for newborns, wherein the control device comprises: (i) a target value providing unit (121) which is configured to provide a target value of the arterial CO2 partial pressure, (ii) a minute volume determination unit (122) which is configured to determine a target value of a minute volume based on the target value of the arterial CO2 partial pressure and a determined value or a value for the arterial CO2 partial pressure derived from the end-tidal CO2 partial pressure if the determined value or the value for the arterial CO2 partial pressure derived from the end-tidal CO2 partial pressure lies outside a first predefined value range around the target value of the arterial CO2 partial pressure or lies within the first predefined value range for a time period less than a predefined time period, and (iii) a respiratory gas source control unit (123) which is configured to receive the target value of the minute volume and to control the respiratory gas source based on the target value of the minute volume. The ventilator according to the invention allows particularly gentle ventilation.

The invention relates to a ventilator for supplying respiratory gas, a control device for controlling a respiratory gas source of a ventilator, a method for supplying respiratory gas and a computer program.

Ventilators for respiratory gas supply are generally known as a life-saving device. However, they also pose various risks for the patient being ventilated, especially if the ventilator is set incorrectly.

In this context, it is known to make ventilator settings depending on patient data or a condition of the patient to be ventilated. However, it is also important to make an adjustment during the ventilation period, for example to take into account the development of the condition of the patient to be ventilated and in particular to react to spontaneous respiratory. For example, the arterial CO2 partial pressure, which can be determined using a blood sample or an electrode on the skin, is known to be used for this purpose. However, both are not unproblematic, especially for infants, as a determination with the help of an electrode must be operated at a certain temperature and this can cause burn damage to the skin. As an alternative, the arterial CO2 partial pressure is therefore partly determined based on the CO2 content of the exhaled air (so-called end-tidal CO2 partial pressure), wherein a higher error tolerance is accepted in this case. The ventilator settings can then be made on the basis of this data.

The ventilator is usually adjusted by setting a respiratory rate and a maximum inspiratory pressure, wherein a closed feedback control can be provided to regulate the settings. However, precise regulation of these values is necessary in order to minimize the risk of damage caused by ventilation, for example due to excessive pressure or excessive respiratory rate, which can particularly affect the lungs. Inaccurate regulation of the ventilator can also lead to CO2 content in the blood being too low, which can entail a risk of damage, particularly to the brain. For children, newborns and especially premature infants, even small deviations from optimum ventilation entail a high risk of damage. For newborns, additionally the lungs significantly change over the period of ventilation, which can extend from several hours to several days, whereby a continuous adaptation is necessary to provide optimum ventilation.

Ventilators are known from the state of the art, for example from U.S. Pat. No. 7,802,571 B2 and EP 1 984 050 B1.

It is therefore the aim of the present invention to provide a solution that compensates for the above-mentioned disadvantages of the prior art and improves the safety of ventilators.

According to a first aspect, a ventilator for supplying respiratory gas is proposed, in particular to newborns. It preferably comprises some or all of the following elements: a respiratory gas source, a control device for controlling the respiratory gas source, a sensor device connected to the control device for detecting an end-tidal CO2 partial pressure, an exchangeable respiratory gas hose with at least a first connecting piece for the respiratory gas hose on the respiratory gas source and a second connecting piece for the respiratory gas hose on a patient interface, such as a mouthpiece, and a user interface which is configured to receive user input. The control device preferably comprises a target value providing unit which is configured to provide a target value of the arterial CO2 partial pressure. The control device preferably comprises a minute volume determination unit which is configured to determine a target value of a minute volume based on the target value of the arterial CO2 partial pressure and a determined value for the arterial CO2 partial pressure or a value for the arterial CO2 partial pressure derived from the end-tidal CO2 partial pressure if the determined value for the arterial CO2 partial pressure or the value for the arterial CO2 partial pressure derived from the end-tidal CO2 partial pressure lies outside a first predefined value range around the target value of the arterial CO2 partial pressure or lies within the first predefined value range for a time period less than a predefined time period. The control device further preferably comprises a respiratory gas source control unit which is configured to receive the target value of the minute volume and to control the respiratory gas source, preferably the fan, based on the target value of the minute volume. The respiratory gas source may be or comprise at least a motor and/or a fan, a valve, bellows, a diaphragm, a pressurized gas device, or a combination of the foregoing. If a fan is mentioned as an example in the following, it should be understood that the other variants are also included.

When the term CO2 partial pressure without further additions or a target value of the CO2 partial pressure is used in the following, this refers preferably to the arterial CO2 partial pressure. This term refers to the location of the target value. No intervention in the patient is required for this and the invention relates only to the device and the control method within the device.

The sensor device for detecting the end-tidal CO2 partial pressure preferably comprises a sensor for direct and/or indirect detection of the end-tidal CO2 partial pressure, wherein the sensor device detects a measured variable during indirect detection of the end-tidal CO2 partial pressure, from which the end-tidal CO2 partial pressure can be calculated. The sensor device is preferably configured to output the detected and/or calculated CO2 partial pressure, preferably to the minute volume determination unit or an cache memory.

A sensor can be arranged directly in the mouthpiece or in the vicinity of the mouthpiece, for example in the respiratory gas hose or a connecting piece to the mouthpiece or the respiratory gas hose, wherein the sensor is positioned such that it is possible to detect the end-tidal CO2 partial pressure in a respiratory gas. A mouthpiece can be, for example, a hose or a respiratory mask. The mouthpiece, i.e. the hose or the respiratory mask, has a respiratory gas outlet through which respiratory gas can be fed into patient's lungs, for example. Preferably, the sensor device, in particular the sensor, is arranged such that the end-tidal CO2 partial pressure is detected between the respiratory gas source and the respiratory gas outlet, preferably between the respiratory gas hose and the respiratory gas outlet, particularly preferably as close as possible to the patient's lungs. The sensor device or sensor is particularly preferably independent of the choice of mouthpiece.

The user interface is preferably an input unit, which preferably comprises a touch screen and/or buttons or switches. Alternatively or additionally, the user interface can also comprise a computer with a screen, a mouse and/or a keyboard. The user interface can be connected to the other components, for example the control device, by means of a cable or wirelessly. If there is a wireless connection between the user interface and the control device, the user interface can preferably also include a tablet, smartphone, etc. The user interface can display a graphical user interface (GUI) with which a user can predefine, change and/or monitor settings.

The control device preferably comprises a target providing unit which is configured to provide a target value of the arterial CO2 partial pressure. The target value of the arterial CO2 partial pressure corresponds to the value of the arterial CO2 partial pressure that is to be reached or maintained during ventilation. The target arterial CO2 partial pressure value is a predefined value that may correspond to a standard arterial CO2 partial pressure value. Preferably, the target value of the arterial CO2 partial pressure is based on experience of the user. Additionally or alternatively, the target value of the arterial CO2 partial pressure can be determined based on patient data and/or based on measurement data. Preferably, the target value of the arterial CO2 partial pressure is greater than 35 mmHg, particularly preferably 40 mmHg, and less than 45 mmHg.

The minute volume determination unit is preferably configured to determine a target value of a minute volume. The minute volume corresponds to a volume of respiratory air that is inhaled and exhaled by a patient per minute. The target value of the minute volume corresponds to the value of the minute volume that is to be provided by the ventilator. The target value of the minute volume is determined based on the target value of the arterial CO2 partial pressure and a determined value for the arterial CO2 partial pressure and/or a value for the arterial CO2 partial pressure derived from the end-tidal CO2 partial pressure. A determined value for the arterial CO2 partial pressure can, for example, be determined from a blood provided sample by a patient. The arterial CO2 partial pressure value derived from the end-tidal CO2 partial pressure is an arterial CO2 partial pressure that is derived from the end-tidal CO2 partial pressure. In particular, the arterial CO2 partial pressure is estimated or approximated by the end-tidal CO2 partial pressure.

In one aspect of the invention, a minute volume is determined only if the determined value for the arterial CO2 partial pressure or the value for the arterial CO2 partial pressure derived from the end-tidal CO2 partial pressure lies outside a first predefined value range around the target value of the arterial CO2 partial pressure and/or lies within the first predefined value range for a time period less than a predefined time period. The predefined time period should be sufficiently long to ensure that the arterial CO2 partial pressure is “regulated out” and is no longer in the transient range. The predefined time period is predefined by a user, for example by entering a time period, preferably via the user interface, or is based on preset values that can be read from a memory, for example. In a preferred embodiment, the predefined time period is 60 s to 100 s. Preferably, the target value of the arterial CO2 partial pressure lies in a middle of the first predefined value range. The first predefined value range may be indicated by a quantity of 2 w, wherein in a case in that the target value of the arterial CO2 partial pressure is in the middle of the first predefined value range, a quantity w corresponds to a deviation from the target value of the arterial CO2 partial pressure. For example (and preferably), the first predefined value range may be given by w=2 mmHg.

The respiratory gas source control unit is preferably configured to receive the target value of the minute volume and to control the respiratory gas source, preferably the fan, based on the target value of the minute volume.

The ventilator according to one aspect of the invention preferably adjusts the minute volume only in cases where the above-mentioned condition is fulfilled, i.e. the minute volume is preferably not adjusted if the above-mentioned condition is not fulfilled. As a result, frequent adjustment of the minute volume is prevented and the minute volume is only adjusted if the determined value for the arterial CO2 partial pressure or the value for the arterial CO2 partial pressure derived from the end-tidal CO2 partial pressure lies outside the first predefined value range and/or lies within the first predefined value range for a time period less than a predefined time period. This enables gentler ventilation by reducing the number of interventions while allowing the arterial CO2 partial pressure to stabilize around the target value without being influenced. This can increase the safety of the ventilator.

In particular, the ventilator according to the invention can provide automatic and continuous adaptation of the ventilator to the patient.

The minute volume determination unit can be realized by a suitable controller, in particular a PI controller, which determines a target value, in particular a further target value, for the minute volume based on the above-mentioned features. “Further target value” means here that a minute volume is already set, i.e. that the ventilator is operated based on a target value for the minute volume, and that the minute volume determination unit is configured to determine a further target value for the minute volume. The minute volume determination unit forwards the further target value for the minute volume to the respiratory gas source control unit. The respiratory gas source control unit is configured to receive the further target value for the minute volume and to control the respiratory gas source, preferably the fan, based on the further target value for the minute volume. This results in a change of the control of the respiratory gas source, preferably the fan, i.e. a control intervention.

The claimed ventilator thus preferably comprises a control device which uses a closed control loop to regulate the arterial CO2 partial pressure during artificial ventilation.

Any data, i.e. in particular the target value of the arterial CO2 partial pressure, a first predefined value range, a second predefined value range, a predefined time period, patient data, etc., which are used for controlling the arterial CO2 partial pressure during artificial ventilation by the ventilator according to the invention, can be provided via a memory or received via an interface, for example the user interface. Patient data can, for example, be read from a patient-specific memory, such as a chip card or a memory in a cloud, and/or entered manually, i.e. via the user interface by a user, or provided to the control device via a connection, in particular a wireless connection.

In an advantageous embodiment of one aspect of the invention, the respiratory gas source control unit is further configured to receive a preset value for the minute volume and to control the respiratory gas source, preferably the fan, based on the preset value of the minute volume, if the determined arterial CO2 partial pressure or the value for the arterial CO2 partial pressure derived from the end-tidal CO2 partial pressure lies within the first predefined value range for the predefined time period and in a second predefined value range around the target value of the arterial CO2 partial pressure after the predefined time period, wherein the second predefined value range is preferably greater than the first predefined value range.

It is advantageous to determine a minute volume only if the determined value for the arterial CO2 partial pressure or the value for the arterial CO2 partial pressure derived from the end-tidal CO2 partial pressure lies outside a first predefined value range or lies within the first predefined value range for a time period that is shorter than the predefined time period. Preferably, a minute volume is not determined if the determined arterial CO2 partial pressure or the value for the arterial CO2 partial pressure derived from the end-tidal CO2 partial pressure lies within the first predefined value range for the predefined time period and, after the end of the predefined time period, within a second predefined value range which is provided around the target value of the arterial CO2 partial pressure. The term “after the predefined time period” refers to a time period which is temporally after the predefined time period, wherein it is preferred that the determined value for the arterial CO2 partial pressure or the value for the arterial CO2 partial pressure derived from the end-tidal CO2 partial pressure lies within the second predefined value range immediately after the predefined time period. The word “within” in relation to the first or second predefined value range refers to the fact that the determined value for the arterial CO2 partial pressure or the value for the arterial CO2 partial pressure derived from the end-tidal CO2 partial pressure lies within the predefined value range, i.e. within predefined limits. Preferably, the target value of the arterial CO2 partial pressure lies in a middle of the second predefined value range. As already indicated above, the target value of the arterial CO2 partial pressure preferably lies in a middle of the first predefined value range. It is particularly preferred that the target value of the arterial CO2 partial pressure lies in a middle of the first predefined value range and a middle of the second predefined value range. The second predefined value range can be indicated by a quantity 2 L, wherein in a case in that the target value of the arterial CO2 partial pressure lies in the middle of the second predefined value range, a quantity L corresponds to a deviation from the target value. The second predefined value range is preferably greater than the first predefined value range, i.e. preferably L>w. Preferably is L=6 mmHg.

As a result, a smaller value range must be fulfilled as a condition if the arterial CO2 partial pressure is far from the target value of the arterial CO2 partial pressure, i.e. outside the first predefined value range, and a larger value range must be fulfilled as a condition if the arterial CO2 partial pressure lies within the first predefined value range. The preferred embodiment thus leads to a further reduction in the number of interventions and can thus contribute to improving the safety of the ventilator.

In another advantageous embodiment of an aspect of the invention, the minute volume determination unit is configured to determine the target value for the minute volume based on the target value of the arterial CO2 partial pressure and a value derived from the end-tidal CO2 partial pressure, wherein the derived value corresponds to a piecewise linear approximation based on the end-tidal CO2 partial pressure.

It was found that at one point in time the arterial CO2 partial pressure can be approximated on the basis of the end-tidal CO2 partial pressure via a linear relationship, i.e. that at one point in time the end-tidal CO2 partial pressure is determined and this is approximated via a linear relationship. Over time, this leads to a piecewise, i.e. partial, linear approximation.

In a preferred variant of the above embodiment, the value derived from the end-tidal CO2 partial pressure is calculated by the following equation:

$$Pa \hat{} CO 2 = C 1 \times PetCO 2 + C 2 ,$$

where Pa\hat{ }CO2 is the value derived from the end-tidal CO2 partial pressure, PetCO2 is the end-tidal CO2 partial pressure and C1 and C2 are predefined parameters. Preferably, the predefined parameters C1 and C2 can be determined depending on patient data and/or can be set, i.e. preset, based on empirical values, for example by a physician. Preferably, initial values can be determined for the predefined parameters C1 and C2 from a blood sample provided, which can then be further adjusted, for example with aid of an algorithm. In one embodiment, is C1=1 or C1 is not equal to 1, in which case it is preferred that C1 is greater than 0.9 and less than 1.1. Alternatively, it can also be assumed that the end-tidal CO2 partial pressure corresponds to the arterial CO2 partial pressure. For example, C2=6.65 mmHg, as also known from Bhat, Y. R.; Abhishek, N.: Mainstream end-tidal carbon dioxide monitoring in ventilated neonates. In: Singapore Medical Journal 49, (2008), March, No. 3, pp. 199-203. A possibility for determining C2 can additionally be found, for example, in U.S. Pat. No. 7,902,571. As an alternative to a linear approximation, an approximation can also be made which takes into account a temperature and/or humidity, preferably in the mouthpiece.

In a further advantageous embodiment of an aspect of the invention, the respiratory gas source control unit is configured to determine a first maximum inspiratory pressure and a first respiratory rate for controlling the respiratory gas source, preferably the fan, based on the determined target value of the minute volume, and to control the respiratory gas source, preferably the fan, based on the first determined maximum inspiratory pressure and the first determined respiratory rate.

The maximum inspiratory pressure corresponds to the maximum pressure generated by the ventilator during an inspiration. The respiratory rate indicates the number of breaths within a certain time period.

In a preferred variant, the respiratory gas source control unit is further configured to receive a measured minute volume, the first determined maximum inspiratory pressure and/or the first determined respiratory rate, to determine a second maximum inspiratory pressure and a second respiratory rate based at least on the measured minute volume, the first determined maximum inspiratory pressure and/or the first determined respiratory rate, and to control the respiratory gas source, preferably the fan, based on the second determined maximum inspiratory pressure and the second determined respiratory rate.

The measured minute volume can be received as a measured value or as a value of the minute volume derived from a measured value. Preferably, the minute volume can be determined via a continuous respiratory gas flow measurement, wherein the minute volume can be determined from the continuous respiratory gas flow. Preferably, a maximum inspiratory pressure is adjusted with a step size of 1 mbar and a respiratory rate is adjusted with a step size of 2/min, i.e. a first maximum inspiratory pressure has a difference of preferably 1 mbar to a second maximum inspiratory pressure and a first respiratory rate has a difference of preferably 2/min to a second respiratory rate. Alternatively, a maximum inspiratory pressure can be selected with a step size of 0.5 mbar and a respiratory rate with a step size of 1/min, so that a slower reaction occurs. However, other step sizes can also be selected.

In particular, the described regulation of the arterial CO2 partial pressure can be understood as a closed control loop, wherein the control loop has an outer loop, which serves to regulate the arterial CO2 partial pressure by the minute volume, and an inner loop, by which the minute volume is adjusted by the maximum inspiratory pressure and the respiratory rate. The outer loop thus provides a value for the inner loop. Preferably, an execution frequency of the outer loop, i.e. the frequency at which the arterial CO2 partial pressure is controlled, is selected from 1/20 Hz to 1/60 Hz and is particularly preferably 1/20 Hz. Furthermore, an execution frequency for the inner control loop, i.e. the frequency with which the minute volume is controlled, is preferably selected from ¼ Hz to 1/Hz and is particularly preferably ¼ Hz.

This type of control, in particular with an inner control loop and an outer control loop, enables a further reduction in interventions, which can further increase the safety of a patient when using the ventilator.

In a further preferred variant, the respiratory gas source control unit is further configured to obtain a PIP value range having a lower PIP limit value and an upper PIP limit value for the maximum inspiratory pressure and/or an RR value range having a lower RR limit value and an upper RR limit value for the respiratory rate, and to control the respiratory gas source, preferably the fan, based on the determined maximum inspiratory pressure and the determined respiratory rate, if the determined maximum inspiratory pressure lies within the PIP value range and/or the determined respiratory rate lies within the RR value range.

In particular, this ensures that the maximum inspiratory pressure and the respiratory rate for setting the minute volume cannot assume values that are potentially harmful to the patient. Preferably, the lower PIP limit value, the upper PIP limit value, the lower RR limit value and/or the upper RR limit value are predefined based on patient data, experience values, guidelines and/or a patient condition. In a preferred embodiment, the lower PIP limit value, the upper PIP limit value, the lower RR limit value and/or the upper RR limit value are predefined in a memory of the control device or can be entered via the user interface. Limit values for the input or setting of the maximum inspiratory pressure and respiratory rate make it possible to minimize the risk of controlling the arterial CO2 partial pressure with regard to the setting of the maximum inspiratory pressure and respiratory rate.

In a preferred further development, the respiratory gas source control unit is further configured to receive patient data, preferably a compliance and/or a resistance, to determine a patient-dependent maximum inspiratory pressure and a patient-dependent respiratory rate based at least on the determined minute volume and the patient data, and to control the respiratory gas source, preferably the fan, based on the patient-dependent maximum inspiratory pressure and the patient-dependent respiratory rate.

Compliance is an elastic volume expansion of the airway, especially the lungs. Resistance can be understood as the airway resistance that air has to overcome as it flows through the airways, especially the lungs.

This means that patient-specific data can be used instead of standardized values for the maximum inspiratory pressure and the respiratory rate, which can lead to particularly advantageous control of the arterial CO2 partial pressure.

In another preferred embodiment, the respiratory gas source control unit is further configured to receive a target tidal volume range Vt having a lower Vt target limit value and an upper Vt target limit value, to determine a tidal volume-dependent maximum inspiratory pressure and a tidal volume-dependent respiratory rate based at least on the determined minute volume and the target tidal volume range Vt, and to control the respiratory gas source, preferably the fan, based on the tidal volume-dependent maximum inspiratory pressure and the tidal volume-dependent respiratory rate.

In this embodiment, a target range for the tidal volume can therefore be taken into account for controlling the arterial CO2 partial pressure, i.e. a target value for the minute volume with the desired tidal volume, i.e. a tidal volume in the target range for the tidal volume, is determined by the minute volume determination unit, received by the respiratory gas source control unit and the respiratory gas source is controlled based on this target value for the minute volume.

Preferably, the lower Vt target limit value and the upper Vt target limit value are not hard limits, i.e. in certain situations, the lower Vt target limit value and the upper Vt target limit value can be exceeded or undercut. This prioritizes the provision of a minute volume with a tidal volume in the target range for the tidal volume.

The above variant can advantageously be configured in such a way that the respiratory gas source control unit is also configured to determine a second tidal volume-dependent maximum inspiratory pressure based at least on the determined minute volume if a measured tidal volume is not within the target tidal volume range Vt, and to control the respiratory gas source based on the second tidal volume-dependent maximum inspiratory pressure.

The measured tidal volume can be a measured value or a value derived from a measured value. For example, a tidal volume can be calculated by measuring the airway flow.

In particular, if a target range of the tidal volume is not reached, i.e. if a measured tidal volume is not within the target tidal volume range, i.e. is less than the lower Vt target limit value or greater than the upper Vt target limit value, the target tidal volume range should be reached again. As the tidal volume cannot be adjusted via the respiratory rate but can be adjusted via the maximum inspiratory pressure, the respiratory rate can correspond to the value already set and only the maximum inspiratory pressure is determined. In this way, the number of interventions can be further reduced.

Preferably, the measured minute volume can also be calculated using the following equation: MV=RR*Vt, where MV is the measured minute volume and Vt is a tidal volume.

Preferably, the tidal volume can also be calculated using the following equation: Vt=(PIP−PEEP)*Crs, where PEEP is a positive end-expiratory pressure and Crs is the compliance. A positive end-expiratory pressure is the pressure that exists in the lungs at the end of expiration.

In a preferred variant of the above embodiment, the control device has at least one predefined mode which can be selected by the user interface, wherein the respiratory gas source control unit is configured to control the respiratory gas source based on the target value of the minute volume using the predefined mode.

A predefined mode comprises predefined values and/or value ranges for the various data mentioned, for example a target arterial CO2 partial pressure value, a first predefined value range, a second predefined value range, a predefined time period, patient data, a lower PIP limit value, an upper PIP limit value, a lower RR limit value, an upper RR limit value, a lower Vt target limit value, an upper Vt target limit value, etc., so that these do not have to be set by the user. This leads to an easier operation for the user, so that a potential number of errors can be reduced. The use of modes can therefore increase the safety of the ventilator.

The above variant can advantageously be implemented such that the respiratory gas source control unit is further configured to obtain in a first mode a PIP value range having a lower PIP limit value and an upper PIP limit value for the maximum inspiratory pressure and an RR value range having a lower RR limit value and an upper RR limit value for the respiratory rate, to obtain a predefined value for the maximum inspiratory pressure and to control the respiratory gas source based on the determined respiratory rate if the determined value for the maximum inspiratory pressure is smaller than the predefined value for the maximum inspiratory pressure, wherein the determined respiratory rate lies within the RR value range.

This provides a mode in which a respiratory rate is prioritized and adjusted and the maximum inspiratory pressure remains unchanged. This is particularly desirable if a minute volume is to be increased. This means that the predefined RR value range of the respiratory rate is first exhausted before the maximum inspiratory pressure is determined and adjusted. This can lead to gentler ventilation and can therefore further improve the safety of the patient being ventilated.

The variant of the first mode can also advantageously be configured such that the respiratory gas source control unit is configured to control the respiratory gas source based on the determined maximum inspiratory pressure and the determined respiratory rate if the determined respiratory rate corresponds to an upper RR limit value of the RR value range.

To achieve the desired target value of the minute volume, the respiratory rate is therefore adjusted until an upper RR limit value of the RR value range is reached and, when the upper RR limit value of the RR value range is reached, the maximum inspiratory pressure is adjusted.

Furthermore, the above variant can advantageously be configured such that the respiratory gas source control unit is configured to control the respiratory gas source based on the determined maximum inspiratory pressure and the determined respiratory rate if the determined value for the maximum inspiratory pressure is greater than the predefined value for the maximum inspiratory pressure.

In particular, in the first mode, if the maximum inspiratory pressure is above the predefined value for the maximum inspiratory pressure and the minute volume provided is to be reduced, the maximum inspiratory pressure is reduced first.

Preferably, the respiratory gas source control unit is further configured to control the respiratory gas source based on the determined respiratory rate if the determined maximum inspiratory pressure corresponds to the predefined value for the maximum inspiratory pressure, wherein the determined respiratory rate lies within the RR value range, and to control the respiratory gas source based on the determined maximum inspiratory pressure if the determined respiratory rate corresponds to a lower RR limit value of the RR value range. The first mode can preferably be provided as a basic mode.

This feature can also enable gentler ventilation for the patient in cases where, for example, a limit value of a predefined value range has been reached, which in turn can lead to a further increase in patient safety.

The above variant can advantageously be implemented such that the respiratory gas source control unit is further configured to obtain in a second mode a PIP value range having a lower PIP limit value and an upper PIP limit value for the maximum inspiratory pressure, an RR value range having a lower RR limit value and an upper RR limit value for the respiratory rate, and a target tidal volume range having a lower Vt target limit value and an upper Vt target limit value. The respiratory gas source control unit is preferably configured to determine a maximum inspiratory pressure based on at least the PIP value range, the RR value range and the target tidal volume range. The respiratory gas source control unit preferably controls the respiratory gas source based on the determined maximum inspiratory pressure if a determined value for the tidal volume is not within the target tidal volume range. The respiratory gas source control unit is also preferably configured to determine a respiratory rate based on at least the PIP value range, the RR value range and the target tidal volume range. The respiratory gas source control unit preferably controls the respiratory gas source based on the determined respiratory rate if the determined value for the tidal volume lies within the target tidal volume range. The second mode can therefore be referred to as volume target mode.

Preferably, in this mode, the patient's compliance measured by the ventilator can also be taken into account, wherein “measured” also means that the compliance can be calculated from measured values, for example by dividing the volume change (tidal volume) by the pressure change (PIP-PEEP). The user can specify a target range for the tidal volume, for example. Preferably, a target range for the tidal volume is calculated as a function of the patient's body weight, wherein in one embodiment a target range of 5 to 7 ml/kg is specified. Depending on the patient's compliance and the positive end-expiratory pressure (PEEP) manually set by the medical staff, a maximum inspiratory pressure and a minute frequency are determined and the respiratory gas source is controlled based on the determined values so that the specified minute volume is prioritized with the target range of the tidal volume. Taking into account a predefined target range for the tidal volume can enable particularly gentle ventilation and thus further increase patient safety.

Preferably, the respiratory gas source control unit is also configured to control the respiratory gas source based on the determined maximum inspiratory pressure and the determined respiratory rate if the determined value for the tidal volume is not within the target tidal volume range and the maximum inspiratory pressure corresponds to a lower or upper PIP limit value of the PIP value range.

In particular, if a target range of the tidal volume is not reached, i.e. if a measured tidal volume is not within the target tidal volume range, i.e. is less than the lower Vt target limit value or greater than the upper Vt target limit value, the target tidal volume range should be reached again. As the tidal volume cannot be adjusted via the respiratory rate but can be adjusted via the maximum inspiratory pressure, the respiratory rate can correspond to the value already set and only the maximum inspiratory pressure is determined. In this way, the number of interventions can be further reduced and patient safety can be further improved.

In addition or alternatively to the first mode and the second mode, one or more further modes may be provided, preferably a third mode, wherein the respiratory gas source control unit is preferably further configured to determine in the third mode the maximum inspiratory pressure and the respiratory rate in dependence on patient data, preferably a compliance, a resistance and/or a dead space, so that a work of respiratory of a patient can be minimized. The third mode may preferably correspond to a mode known from the following publications: Otis A B, Fenn W O, Rahn H. Mechanics of respiratory in man. J Appl Physiol 1950; 2:592-607 and Tehrani F T. Method and apparatus for controlling an artificial respirator. U.S. Pat. No. 4,986,268, Issued Jan. 22, 1991.

In a second aspect of the invention, a control device for controlling a respiratory gas source of a ventilator is proposed. The control device is preferably provided for controlling a respiratory gas source of a ventilator, in particular for newborns, wherein the control device preferably comprises a target value providing unit which is configured to provide a target value of the arterial CO2 partial pressure, a minute volume determination unit, which is configured to determine a target value of a minute volume based on the target value of the arterial CO2 partial pressure and a determined value for the arterial CO2 partial pressure or a value for the arterial CO2 partial pressure derived from the end-tidal CO2 partial pressure if the determined value for the arterial CO2 partial pressure or the value for the arterial CO2 partial pressure derived from the end-tidal CO2 partial pressure lies outside a first predefined value range around the target value of the arterial CO2 partial pressure or lies within the first predefined value range for a time period less than a predefined time period. The control device preferably comprises a respiratory gas source control unit configured to receive the target value of the minute volume and to control the respiratory gas source based on the target value of the minute volume. In other variants, the control device can be configured to interact with existing respiratory gas source control units.

In a third aspect of the invention, a method is proposed, namely a method for respiratory gas supply, in particular of newborns, comprising (i) detecting an end-tidal CO2 partial pressure, (ii) providing a target value of the arterial CO2 partial pressure, (iii) determining a target value of a minute volume based on the target value of the arterial CO2 partial pressure and a determined value for the arterial CO2 partial pressure or a value for the arterial CO2 partial pressure derived from the end-tidal CO2 partial pressure, if the determined value for the arterial CO2 partial pressure or the value for the arterial CO2 partial pressure derived from the end-tidal CO2 partial pressure lies outside a first predefined value range around the target value of the arterial CO2 partial pressure or lies within the first predefined value range for a time period less than a predefined time period, (iv) receiving the target value of the minute volume and (v) triggering the respiratory gas source based on the target value of the minute volume.

According to a further aspect of the invention, a computer program is proposed comprising program means for causing a control device according to one of the above-described embodiments of a control device to perform the steps of the method according to one of the above-described preferred embodiments of a method when the computer program is executed on the control device.

The computer program may be provided, stored and/or distributed on a suitable storage medium, such as an optical storage medium or a non-volatile electronic storage medium. It may also be provided together with or as part of a hardware component. The computer program may also be provided by other means, such as via the Internet or via wired or wireless telecommunication means.

Features of advantageous embodiments of the invention are defined in particular in the sub-claims, further advantageous features, designs and embodiments for the person skilled in the art also being apparent from the above explanation and the following discussion.

In the following, the present invention is further illustrated and explained with reference to embodiments shown in the figures.

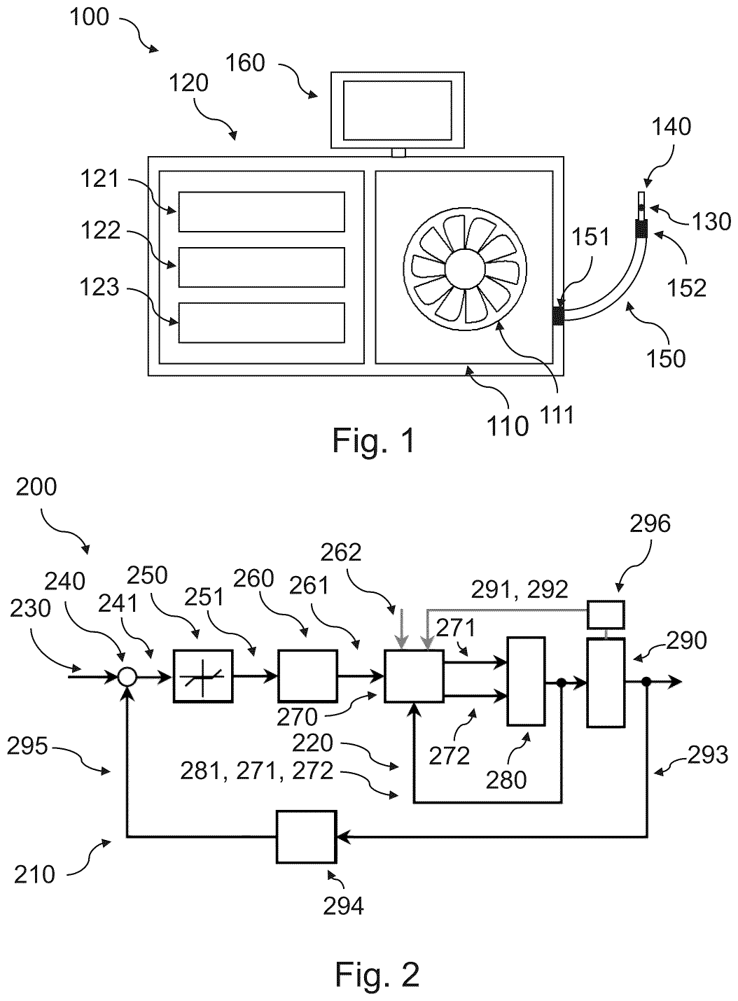

FIG. 1 shows a schematic diagram illustrating a first embodiment of the ventilator according to the invention,

FIG. 2 shows a block diagram of a control circuit that can be used to control the ventilator according to the invention,

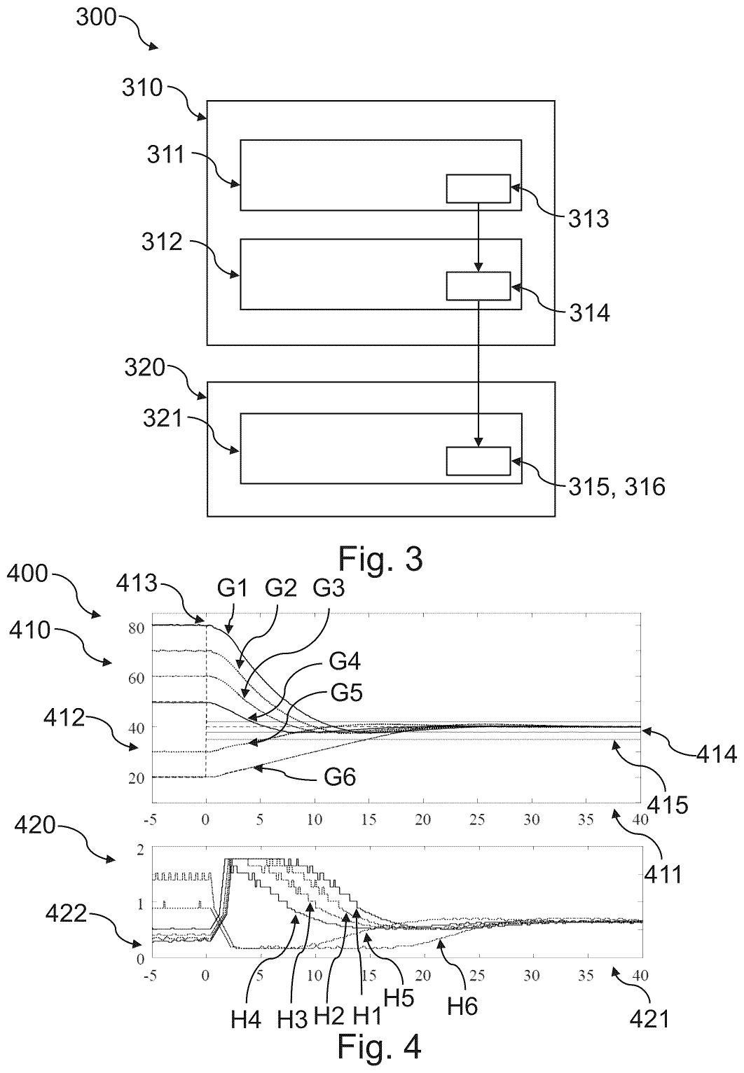

FIG. 3 shows a schematic representation of a hierarchy of the control structure that can be used to control the ventilator according to the invention,

FIG. 4 shows an example of a simulation of the time course of an arterial CO2 partial pressure and a minute volume,

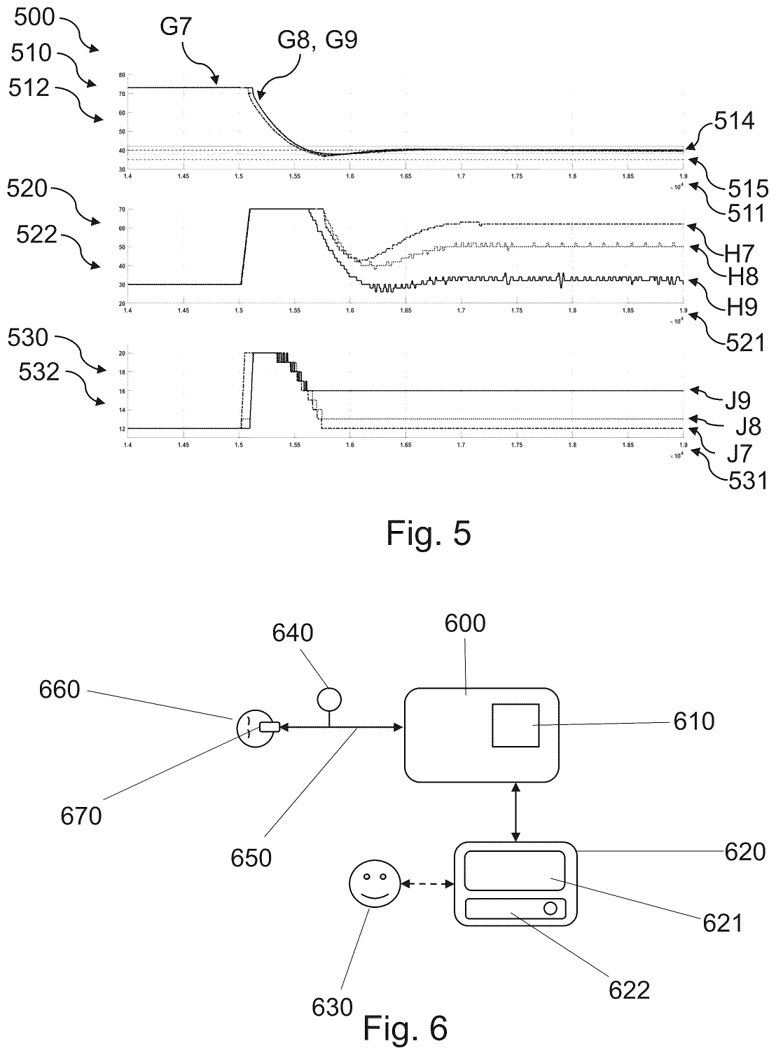

FIG. 5 shows an example of a simulation of the time course of an arterial CO2 partial pressure, a respiratory rate and a maximum inspiratory pressure,

FIG. 6 shows a schematic diagram illustrating a further embodiment of the ventilator according to the invention,

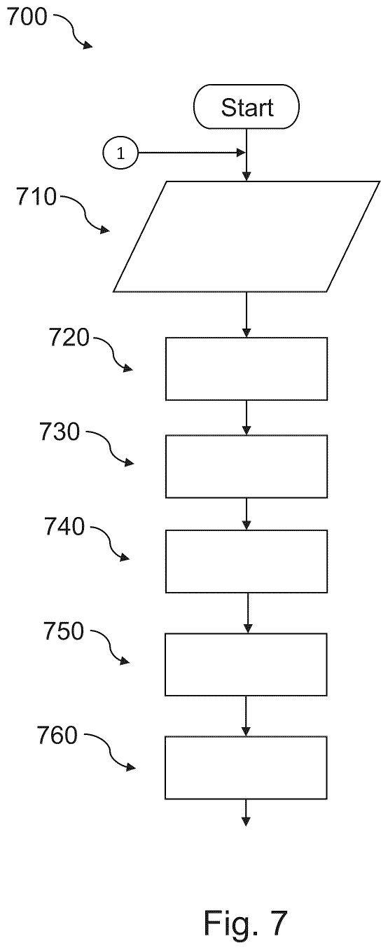

FIG. 7 shows a first part of a schematic flow chart of a first embodiment of the method according to the invention,

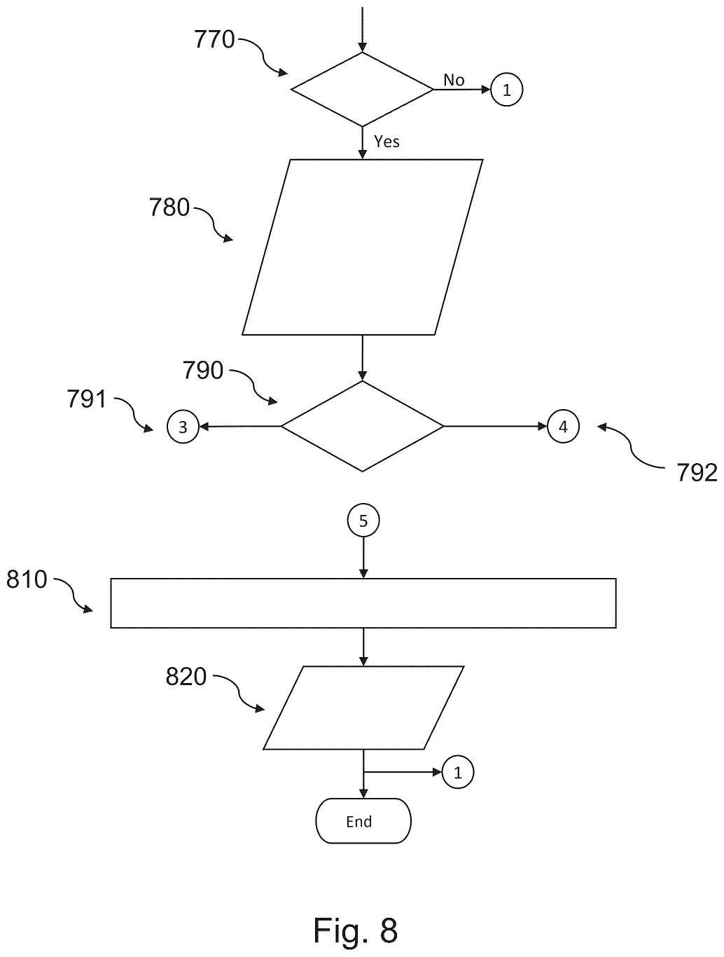

FIG. 8 shows a second part of the schematic flow chart of the first embodiment of the method according to the invention,

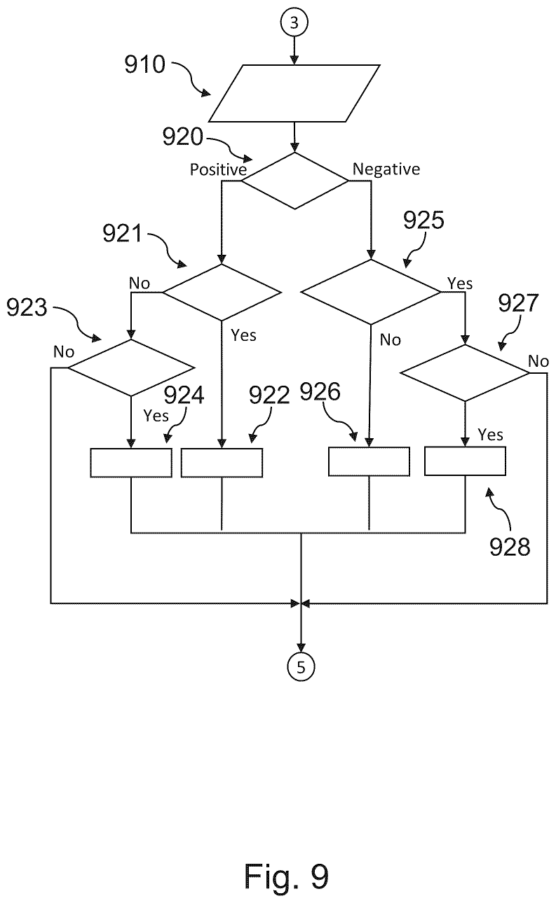

FIG. 9 shows a third part of the schematic flow chart of the first embodiment of the method according to the invention,

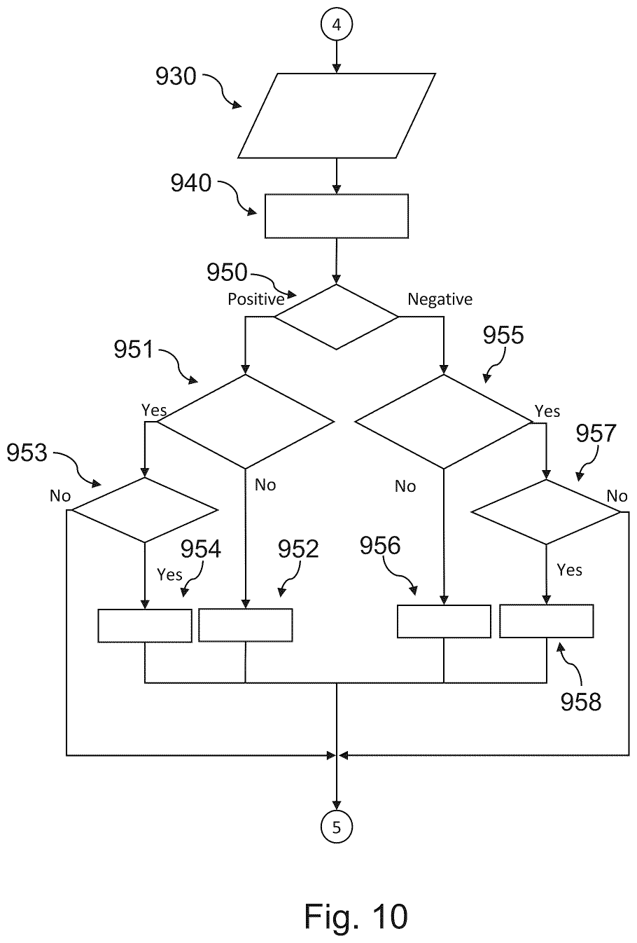

FIG. 10 shows a fourth part of the schematic flow chart of the first embodiment of the method according to the invention, and

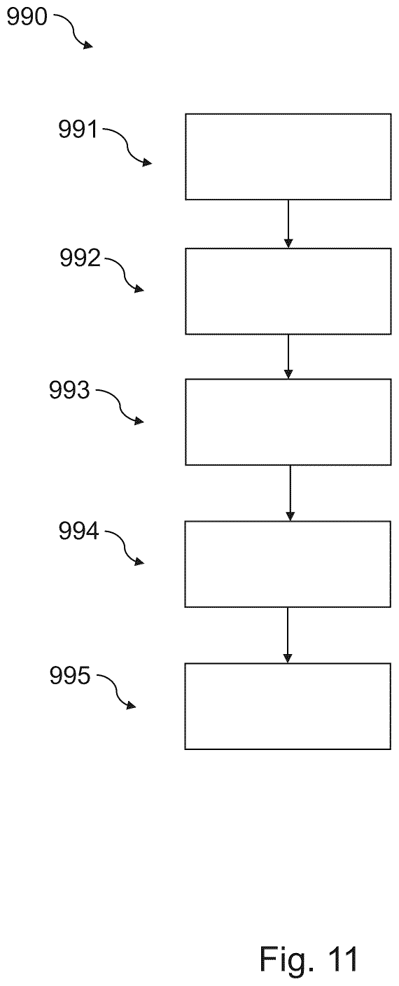

FIG. 11 shows a schematic flow diagram of a second embodiment of the method according to the invention.

FIG. 1 shows a schematic diagram illustrating a first embodiment of the ventilator according to the invention. The ventilator 100 for supplying respiratory gas comprises a respiratory gas source 110, which is exemplarily configured here with a fan 111, and a control device 120 for controlling the respiratory gas source 110. Instead of the fan 111, other units supplying respiratory gas can also be used. In addition, the ventilator 100 comprises a sensor device 130, which is connected to the control device. The sensor device 130 is configured to detect an end-tidal CO2 partial pressure, preferably at a mouthpiece 140. The sensor device 130 then provides signals representing a detected end-tidal CO2 partial pressure to the control device 120.

The ventilator 100 may further comprise a respiratory gas hose 150 having at least a first connecting piece 151 on the respiratory gas source 110 and a second connecting piece 152 for the respiratory gas hose 150 on the mouthpiece 140.

In addition, the ventilator 100 can comprise a user interface 160, which is configured to receive user inputs that can be entered by a user. Furthermore, the user interface 160 is configured to forward the user input or information derived from the user input to the control device 120.

The control device 120 comprises a target value providing unit 121, a minute volume determination unit 122 and a respiratory gas source control unit 123, which can specifically be referred to here as a ventilator control unit 123. The target value providing unit 121 is configured to provide a target value of the arterial CO2 partial pressure. The target value providing unit 121 is further configured to provide the target value of the arterial CO2 partial pressure and the minute volume determination unit 122. The minute volume determination unit 122 is configured to determine a target value of a minute volume based on the target value of the arterial CO2 partial pressure and a determined value for the arterial CO2 partial pressure or a value for the arterial CO2 partial pressure derived from the end-tidal CO2 partial pressure. The minute volume determination unit 122 is further configured to determine a minute volume, in particular only if the determined value for the arterial CO2 partial pressure or the value for the arterial CO2 partial pressure derived from the end-tidal CO2 partial pressure lies outside a first predefined value range around the target value of the arterial CO2 partial pressure or lies within the first predefined value range over a time period that is smaller than a predefined time period. The minute volume determination unit 122 is also configured to forward the target value for the minute volume to the respiratory gas source control unit 123. The respiratory gas source control unit 123 is configured to receive the target value of the minute volume and to control the fan 111 based on the target value of the minute volume.

Preferably, the respiratory gas source control unit 123 is further configured to receive a preset value for the minute volume and to control the fan 111 based on the preset value of the minute volume, if the determined arterial CO2 partial pressure or the value for the arterial CO2 partial pressure derived from the end-tidal CO2 partial pressure lies within the first predefined value range over the predefined time period, and lies also within a second predefined value range around the target value of the arterial CO2 partial pressure after the predefined time period. In this case, the second predefined value range is greater than the first predefined value range. Preferably, the first and the second predefined value range are arranged around the same mean value, preferably the target value of the arterial CO2 partial pressure.

Further preferably, the respiratory gas source control unit 123 is configured to determine a first maximum inspiratory pressure and a first respiratory rate for controlling the fan 111 based on the determined target value of the minute volume, and to control the fan 111 based on the first determined maximum inspiratory pressure and the first determined respiratory rate. Furthermore, the respiratory gas source control unit 123 is configured to receive a measured minute volume, the first determined maximum inspiratory pressure and/or the first determined respiratory rate, to determine a second maximum inspiratory pressure and a second respiratory rate based at least on the measured minute volume, the first determined maximum inspiratory pressure and/or the first determined respiratory rate, and to control the fan 111 based on the second determined maximum inspiratory pressure and the second determined respiratory rate. Such control via a control loop is also shown in FIG. 2.

FIG. 2 shows a block diagram 200 of a control circuit that can be used to control the ventilator 100 according to the invention. The control circuit 200 comprises an outer control circuit 210 and an inner control circuit 220. A reference variable 230 here comprises a target value of the arterial CO2 partial pressure. The control preferably comprises a comparison 240 of the target value of the arterial CO2 partial pressure with a value for the arterial CO2 partial pressure derived from the end-tidal CO2 partial pressure. Such an estimate is preferably based on a linear approximation from the end-tidal CO2 partial pressure in the respiratory gas.

The error 241 resulting from the comparison 240 can first be filtered by an adaptive dead zone element 250. This filter provided in the control loop enables a minute volume determination as follows: If the dead zone is not active, i.e. if the arterial CO2 partial pressure lies outside 2 w or lies within 2 w but a predefined time period T has not yet been reached, a minute volume is calculated based on the difference between the target value of the arterial CO2 partial pressure and the value for the arterial CO2 partial pressure derived from the end-tidal CO2 partial pressure, wherein a derivation is made in particular by an estimate, and forwarded to the respiratory gas source control unit. If the dead zone is reached, i.e. the arterial CO2 partial pressure was within the first predefined value range 2 w over a predefined time period T and the arterial CO2 partial pressure lies within the second predefined value range 2 L, then preferably no new minute volume is calculated but the previously used minute volume is forwarded to the respiratory gas source control unit.

The filtered error 251 is forwarded to a controller 260, preferably a PI controller. The controller converts the filtered error 251 into a target value for the minute volume 261. In particular, the dead zone element 250 and the controller 260 together can be understood as a minute volume determination unit, for example the minute volume determination unit 122. The target value of the minute volume 261 is then forwarded to a respiratory gas source control unit 270, which may correspond, for example, to the respiratory gas source control unit 123 and may in turn be referred to as the ventilator control unit 270. The respiratory gas source control unit 270 is configured to determine a first maximum inspiratory pressure 271 and a first respiratory rate 272 and to forward them to the fan 280, which may, for example, correspond to the fan 111. Further preferably, the respiratory gas source control unit 270 may be configured to obtain a measured minute volume 281, a first determined maximum inspiratory pressure 271 and a first determined respiratory rate 272 and to determine a second maximum inspiratory pressure and a second respiratory rate based on at least the measured minute volume 281, the first determined maximum inspiratory pressure 271 and the first determined respiratory rate 272. Preferably, the second maximum inspiratory pressure or the second respiratory rate can be understood as any further determined maximum inspiratory pressure or any further determined respiratory rate. In a further variant, the ventilation control unit may also be configured to take into account a dead space 262, which represents the space of the respiratory system that is not involved in pulmonary gas exchange, in order to determine a maximum inspiratory pressure and a respiratory rate. In particular, other parameters of a patient 296 can also be used, for example a compliance 291 and/or a resistance 292 of the patient 296.

The fan 280 uses the values of the first maximum inspiratory pressure and the first respiratory rate or the second maximum inspiratory pressure and the second respiratory rate to use the ventilator, i.e. to provide respiratory air through the mouthpiece 290 to a patient 296. In addition, an end-tidal CO2 partial pressure 293 is preferably detected at the mouthpiece 290 by a sensor device when the ventilator 100 is used and used for estimating 294 the arterial CO2 partial pressure 295. Thus, closed-loop ventilation with automatic control of the arterial CO2 partial pressure is provided.

FIG. 3 shows a schematic representation of a hierarchy of the control structure that can be used to control the ventilator 100 according to the invention. In particular, the control structure 300 comprises a cascaded controller 310 and a fan 320 corresponding, for example, to the fan 111. The cascaded controller 31 comprises an outer control loop 311 and an inner control loop 312. The outer control loop 311 represents the master controller that maintains adequate gas exchange. The inner control circuit 312 represents the middle controller and aims to ensure safe ventilation. A lower control circuit 321 comprises the specific control of the fan for performing ventilation, i.e., for example, a control of the ventilation with respect to pressure, flow rate and volume of the ventilation gas. According to the invention, an arterial CO2 partial pressure 313 in the outer control circuit is converted into a minute volume 314 in the inner control circuit 312, which in turn is converted into a maximum inspiratory pressure 315 and a respiratory rate 316.

FIG. 4 shows an example of a simulation 400 of a time course of an arterial CO2 partial pressure and a minute volume. In particular, FIG. 4 shows a diagram 410 in which a time in minutes is indicated on the horizontal axis 411 and an arterial CO2 partial pressure in mmHg is indicated on the vertical axis 412. Furthermore, FIG. 4 shows a diagram 420 in which a time in minutes is indicated on the horizontal axis 421 and a minute volume in liters is indicated on the vertical axis 422. The diagrams show simulated step responses at different levels of hypercapnia and hypocapnia, i.e. increased and decreased CO2 partial pressure, respectively. Each line G1, G2, G3, G4, G5, G6 (in diagram 410 in the left area from top to bottom) represents the course of the arterial partial pressure in an experiment and each line H1, H2, H3, H4, H5, H6 (in diagram 420 associated with the respective line G1 to G6) represents a corresponding minute volume. At t=0 min, a jump of the reference signal from the respective stationary value to 45 mmHg is applied, marked by the vertical dashed line 413. The shaded area 414, which is marked by small dots and in which the various lines G1 to G6 have reached after 20 minutes at the latest in this example, marks the adaptive dead zone, i.e. the target zone. The thick dotted line 415 stands for an imaginary 35 mmHg threshold, which must not be exceeded due to controller interventions, e.g. when falling below it. In particular, the course of the arterial CO2 partial pressure after entering a changed target value of the arterial partial pressure shows a leveling off of the arterial CO2 partial pressure in the dead zone. In general, a leveling off is faster in hypercapnia (G1-G4) than in hypocapnia (G5, G6) due to the lower limit of the manipulated variables, which are reached faster and longer.

FIG. 5 shows an example of a simulation 500 of a time course of an arterial CO2 partial pressure, a respiratory rate and a maximum inspiratory pressure. In particular, FIG. 5 shows a diagram 510 in which a time in seconds is indicated on the horizontal axis 511 and an arterial CO2 partial pressure in mmHg is indicated on the vertical axis 512. Furthermore, FIG. 5 shows a diagram 520 in which a time in seconds is indicated on the horizontal axis 521 and a respiratory rate RR per minute is indicated on the vertical axis 522. In addition, FIG. 5 shows a diagram 530 in which a time in seconds is indicated on the horizontal axis 531 and a maximum inspiratory pressure in mbar is indicated on the vertical axis 532.

In diagram 510, a dead time range is again shown as a shaded area with small dots, which can also be understood as the target range for the arterial CO2 partial pressure. Furthermore, a lower limit is shown in diagram 510 as a long dashed line 515. In the diagrams 510, 520 and 530, two modes of the ventilator are shown, the first mode being characterized by the graphs G7, H7, J7, the second mode by the graphs G8, H8, J8, and the third mode by the graphs G9, H9, J9. The time course of the arterial CO2 partial pressure in the third mode corresponds approximately to the time course of the arterial CO2 partial pressure in the second mode, taking into account the accuracy of the diagram, so that the time course of the arterial CO2 partial pressure in the third mode, which serves primarily as a reference, cannot be seen in diagram 510.

First, the model is ventilated to a steady-state hypercapnia of 73 mmHg by ventilating with a maximum inspiratory pressure of 12 mmHg and a respiratory rate of 30 per minute for 15 000 s (not shown in the figure). In this condition, the controller is activated at t=15 000 s with moderate parameterization. The response time is similar in all three modes, although the response time in the first mode is faster at 505 s than the response time of 545 s in the second and third modes. In particular, the faster response time of the first mode is due to the fact that both output parameters, i.e. the maximum inspiratory pressure and the respiratory rate, can be changed in the first mode, while only one parameter is changed in the second mode and in the third mode. All three modes lead to undershoots between 36.5 and 37.5 mmHg and are within the target range. However, the ventilation parameters, i.e. the maximum inspiratory pressure and the respiratory rate at which the target range is reached, differ significantly, as can be seen from diagrams 520 and 530.

After setting the maximum inspiratory pressure and respiratory rate to the maximum, i.e. corresponding to the upper PIP limit value and the upper RR limit value, to reduce the arterial CO2 partial pressure in all modes, different combinations of maximum inspiratory pressure and respiratory rate occur. For example, the first mode (G7, H7, J7) shows a combination of highest respiratory rate and minimum possible maximum inspiratory pressure. In contrast, the reference, i.e. in the third mode (G9, H9, J9), stabilizes at a significantly higher maximum inspiratory pressure and a lower respiratory rate. The second mode lies in between. The output values of the third and second modes correspond to their settings. For the second mode, the settings include a selection of the maximum inspiratory pressure according to predefined compliance and volume targets. For the reference, i.e. the third mode, this means not lowering the maximum inspiratory pressure below a prioritization threshold of 16 mbar, if possible. The parameters that ultimately result in these two modes therefore depend on the settings that the user has preset.

FIG. 6 shows a schematic diagram illustrating a further embodiment of the ventilator according to the invention. In FIG. 6, the ventilator 600 is equipped with a control device 610, which is integrated in the ventilator 600. However, the control device 610 can alternatively also be provided outside the ventilator 600 and connected to the ventilator 600 via wired or wireless communication. The ventilator 600 comprises a user interface 620. The user interface 620 may be provided as part of the ventilator 600 or separately from the ventilator 600. In this exemplary embodiment, the user interface 620 comprises a display 621 and an input device 622, which may comprise, for example, a touch screen, buttons, a mouse and/or a keyboard. Via the input device 622, a user 630 can enter data and preferably adjust and/or monitor the controllers and the ventilator 600 via the user interface 620. Preferably, the ventilator 600 comprises a sensor or sensor device 640 for detecting an end-tidal CO2 partial pressure provided in a ventilator circuit 650. The sensor or the sensor device 640 can be provided in a main flow or a bypass flow of the respiratory air. A patient 660 can be connected to the ventilator 600 via a mouthpiece or a hose 670 with the ventilator according to the invention. The control device 610 is configured to provide a target value for an arterial CO2 partial pressure. The control device is further configured to determine a target value of a minute volume based on the target value of the arterial CO2 partial pressure and a determined value for the arterial CO2 partial pressure or a value for the arterial CO2 partial pressure derived from the end-tidal CO2 partial pressure, if the determined value for the arterial CO2 partial pressure or the value for the arterial CO2 partial pressure derived from the end-tidal CO2 partial pressure lies outside a first predefined value range around the target value of the arterial CO2 partial pressure or lies within the first predefined value range for a time period less than a predefined time period. Furthermore, the control device 610 is configured to receive the target value of the minute volume and to control a fan based on the target value of the minute volume.

FIG. 7 shows a first part of a schematic flowchart of a first embodiment of the method according to the invention. In particular, the method 700 may comprise, in a first step 710, a reading of useful inputs with a target value for an arterial CO2 partial pressure PaCO2 target and preferably values for the parameters C1 and C2. In addition, a measured value for an end-tidal CO2 partial pressure and a minute volume can preferably be read in. In a second step 720, a value Pa\hat{ }CO2 derived from the end-tidal CO2 partial pressure can then preferably be calculated. In a third step 730, an error e (PaCO2)=PaCO2-target−Pa\hat{ }CO2 can then be calculated. In a fourth step 740, a filtered error e\hat{ }PaCO2 is then calculated, as already described above. In a fifth step 750, a target value of the minute volume MVTarget is then calculated, wherein an error of the minute volume eMV=MVTarget−MV can then preferably be calculated in a sixth step.

FIG. 8 shows a second part of the schematic flow chart of the first embodiment of the method according to the invention. Preferably, in a next step 770, shown in FIG. 8, an amount of the error of the minute volume |eMV| is calculated. If the amount of the error of the minute volume is not greater than zero, the method according to the invention starts from the beginning with the above-mentioned steps. If an amount of the minute volume error is greater than zero, i.e. |eMV|>0, the method proceeds to a next step 780. In step 780, user data is read in, for example a minute volume controller mode, a lower RR limit value and an upper RR limit value for a respiratory rate, and/or a lower PIP limit value and an upper PIP limit value for a maximum inspiratory pressure. In addition, in step 780, current settings of the ventilator such as the current respiratory rate, the current maximum inspiratory pressure and the PEEP are preferably read in. In a next step 790, a mode can then be selected, in particular a first mode 791, which can be understood as a basic mode, or a second mode 792, which can be understood as a volume target mode.

By means of the respective mode, which are described in more detail below in connection with FIGS. 9 and 10, a newly calculated target value for the respiratory rate and the maximum inspiratory pressure can then be checked in a step 810 for exceeding the safety limits, i.e. for exceeding the lower RR limit value and/or an upper RR limit value for the respiratory rate, and/or the lower PIP limit value and the upper PIP limit value for the maximum inspiratory pressure. Preferably, in step 810, the newly calculated target values are adjusted if the lower RR limit value or the lower PIP limit value is undershot and/or the upper RR limit value or the upper PIP limit value is exceeded. In a step 820, new values for the maximum inspiratory pressure and the respiratory rate are then written and preferably output to the respiratory gas source control unit for controlling the fan. After writing the new values for the maximum inspiratory pressure and the respiratory rate, the method 700 can then start again from the beginning, i.e. with step 710.

Preferably, the respiratory rate and the maximum inspiratory pressure can be adjusted in fixed increments or in each case using a suitable controller, e.g. a discrete PI controller, which uses an increment range set by a user.

FIG. 9 shows a third part of the schematic flow chart of the first embodiment of the method according to the invention. In particular, FIG. 9 shows the method for the first mode described above, i.e. the basic mode. In a first step 910 in the first mode, user inputs comprising at least one predefined value for the maximum inspiratory pressure PIPprio are read in. In a step 920, it is checked whether an error of the minute volume eMV is positive or negative. If an error of the minute volume eMV is positive, it is checked in a step 921 whether a respiratory rate RR corresponds to an upper RR limit value RRmax. If the respiratory rate RR is less than the upper RR limit value RRmax, i.e. RR<RRmax, the respiratory rate RR is increased in a step 922 and the next step is step 820, i.e. new values for the maximum inspiratory pressure and the respiratory rate are written and used to control the fan. If the respiratory rate RR is not less than the upper RR limit value RRmax, it is checked in a step 923 whether the maximum inspiratory pressure PIP is less than an upper PIP limit value PIPmax. If the maximum inspiratory pressure PIP is less than the upper PIP limit value, i.e. PIP<PIPmax, the maximum inspiratory pressure is increased in a step 924 and step 820 is then executed, i.e. new values for the maximum inspiratory pressure and the respiratory rate are written and used to control the fan. If the maximum inspiratory pressure PIP is not less than the upper PIP limit value, step 820, which has already been described above, is subsequently executed and new values for the maximum inspiratory pressure and the respiratory rate are preferably written and used to control the fan.

If an error of the minute volume eMV is negative, it is checked in a step 925 whether a maximum inspiratory pressure PIP is greater than the predefined value for the maximum inspiratory pressure PIPprio, i.e. PIP>PIPprio or whether the respiratory rate RR corresponds to the lower RR limit value, RR==RRmin. If none of the above conditions is met, the respiratory rate RR is lowered in a step 926 and the next step is step 820, i.e. new values for the maximum inspiratory pressure and the respiratory rate are written and used to control the fan. If one of the above-mentioned conditions is fulfilled, it is checked in a step 927 whether the maximum inspiratory pressure PIP is greater than the lower PIP limit value PIPmin, i.e. whether PIP>PIPmin applies. If the maximum inspiratory pressure PIP is greater than the lower PIP limit value PIPmin, the maximum inspiratory pressure is reduced in a step 928 and the next step is step 820, i.e. new values are written for the maximum inspiratory pressure and the respiratory rate and used to control the fan. If the maximum inspiratory pressure PIP is not greater than the lower PIP limit value PIPmin, the next step is step 820, i.e. new values for the maximum inspiratory pressure and the respiratory rate are written and used to control the fan.

FIG. 10 shows a fourth part of the schematic flow chart of the first embodiment of the method according to the invention. In particular, FIG. 10 shows the method for the second mode described above, i.e. the volume-target mode. In a first step 930 in the second mode, user inputs are read in, in particular a preferred lower target limit value for the tidal volume Vtpl and a preferred upper target limit value for the tidal volume Vtpu. Preferably, a measured value of the patient's compliance Crs is also read in in step 930. In a next step 940, a target value for a tidal volume Vt is read in or calculated. In a step 950, it is checked whether an error of the minute volume eMV is positive or negative. If an error of the minute volume eMV is positive, it is checked in a step 951 whether the tidal volume Vt is smaller than the preferred lower target limit value Vtpl, and whether the maximum inspiratory pressure PIP is smaller than the upper PIP limit value, i.e. whether Vt<Vtpl and PIP<PIPmax applies, or whether a respiratory rate RR is equal to an upper RR limit value, i.e. whether RR==RRmax applies. If none of the aforementioned conditions is met, the respiratory rate is increased in a step 952 and step 820 is subsequently executed, i.e. new values for the maximum inspiratory pressure and the respiratory rate are written and used to control the ventilator. If one of the aforementioned conditions is met, i.e. (Vt<Vtpl and PIP<PIPmax) or RR==RRmax, it is checked in a step 953 whether a maximum inspiratory pressure PIP is less than an upper PIP limit value PIPmax, i.e. whether PIP<PIPmax applies. If the maximum inspiratory pressure PIP is less than the upper PIP limit value PIPmax, the maximum inspiratory pressure PIP is increased in a step 954 and step 820 is then executed, i.e. new values for the maximum inspiratory pressure and the respiratory rate are written and used to control the fan. If the maximum inspiratory pressure PIP is not less than the upper PIP limit value PIPmax, the next step is step 820, i.e. new values for the maximum inspiratory pressure and the respiratory rate are written and used to control the fan. If an error of the minute volume eMV is negative when checking in step 950, it is checked in a step 955 whether the tidal volume Vt is greater than a preferred upper target limit value for the tidal volume Vtpu and whether a maximum inspiratory pressure PIP is greater than a lower PIP limit value PIPmin, i.e. whether Vt>Vtmax. i.e. whether Vt>Vtpu and PIP>PIPmin applies, or whether a respiratory rate is equal to a lower RR limit value, i.e. whether RR==RRmin applies. If none of the above conditions is met, the respiratory rate is lowered in a step 956 and then step 820 is executed, i.e. new values for the maximum inspiratory pressure and the respiratory rate are written and used to control the fan. If one of the aforementioned conditions is fulfilled, i.e. (Vt>Vtpu and PIP>PIPmin) or RR==RRmin, it is checked in a step 957 whether the maximum inspiratory pressure PIP is greater than a lower PIP limit value PIPmin, i.e. whether PIP>PIPmin applies. If the maximum inspiratory pressure PIP is greater than the lower PIP limit value PIPmin, the maximum inspiratory pressure is reduced in a step 958 and the next step is step 820, i.e. new values are written for the maximum inspiratory pressure and the respiratory rate and used to control the fan. If the maximum inspiratory pressure PIP is not greater than the lower PIP limit value PIPmin, the next step is step 820, i.e. new values for the maximum inspiratory pressure and the respiratory rate are written and used to control the fan.

It should be noted that the preferred lower and upper target limit values Vtpl and Vtpu, in contrast to the limit values PIPmin, PIPmax, RRmin and RRmax, are not hard limits, but limits of a target range that should be adhered to, but which can also be exceeded if more ventilation is required.

FIG. 11 shows a schematic flow chart of a second embodiment of the method according to the invention. In particular, the method according to the invention is a method 990 for supplying respiratory gas, in particular to newborns, comprising a first step 991 of detecting an end-tidal CO2 partial pressure. In addition, the method 990 comprises a second step 992 of providing a target value of the arterial CO2 partial pressure. A third step 993 of the method 990 then comprises determining a target value of a minute volume based on the target value of the arterial CO2 partial pressure and a determined value for the arterial CO2 partial pressure or a value for the arterial CO2 partial pressure derived from the end-tidal CO2 partial pressure, if the determined value for the arterial CO2 partial pressure or the value for the arterial CO2 partial pressure derived from the end-tidal CO2 partial pressure lies outside a first predefined value range around the target value of the arterial CO2 partial pressure or lies within the first predefined value range for a time period less than a predefined time period. Preferably, the method 990 comprises, in a step 994, receiving the target value of the minute volume and, in a step 995, controlling the ventilator based on the target value of the minute volume.

Even if various aspects or features of the invention are shown in combination in the figures, it is apparent to the skilled person-unless otherwise stated-that the combinations shown and discussed are not the only possible ones. In particular, corresponding units or feature complexes from different embodiments can be interchanged with one another.

In implementations of the invention, individual components, e.g. a processor, can take over the functions of various elements mentioned in the claims in whole or in part. Sequences or processes can be implemented as program means of a computer program and/or as special hardware components.

Further considerations on aspects of the invention follow.

The invention comprises an algorithmic method and its implementation in software for closed-loop ventilation, preferably of newborns, with the aim of automatically regulating the arterial CO2 partial pressure PaCO2 while at the same time protecting the lungs during artificial ventilation, preferably of newborns. The algorithm preferably uses a cascaded control loop structure, as shown in FIG. 2, where the outer loop is used to control the PaCO2 by the minute volume MV, while in the inner loop the MV is set by the ventilation parameters Respiratory Rate RR, i.e. respiratory rate, and Peak Inspiratory Pressure PIP, i.e. maximum inspiratory pressure. The execution frequency is preferably 1/20 Hz for the outer control loop and ¼ Hz for the inner control loop.

Preferably, a cascaded control loop is provided for the invention: First, a minute volume is preferably determined in a first time step, then the minute volume is kept constant for a second and further time steps in which only a respiratory rate, a maximum inspiratory pressure and/or a tidal volume are adjusted.

The regulation of PaCO2 is preferably based on the comparison of an individual PaCO2 target specified by medical staff with an estimate, PaĈO2, which can be determined linearly from the end-tidal CO2 partial pressure PetCO2 in the respiratory gas according to the equation PaĈO2=C1*PetCO2+C2. Preferably, PetCO2 can be measured non-invasively via a CO2 sensor, preferably at the patient's mouthpiece, and the parameters C1 and C2 of the equation mentioned can preferably be adjusted by the medical staff during the runtime of the algorithm.

The error e resulting from a comparison of the aforementioned values is preferably first filtered by an adaptive dead zone element (dead zone) before it can be converted into the MV target by a suitable controller, for example a PI controller, wherein P and I can be set variably. The dead zone element primarily enables gentler ventilation by reducing the number of interventions in the steady state while at the same time allowing the controller to settle around the target value in the transient. This is achieved by the dead zone element preferably outputting a filtered error ē=0 precisely when the deviation between the target value and the estimate e has been within a target range of the width ±w around the target value specified by the user for a defined time T. The dead zone element preferably outputs ē=e before the above condition is fulfilled for the first time and after the absolute error has once been greater than a parameter L that can be set by the user, i.e. |e|>L. Preferably, the specified target range can be set to 4 mmHg.

The MV target specified by the outer control loop is preferably converted into specific values for RR and PIP in the inner control loop by a ventilation controller, wherein the user can choose from various lung-protecting modes, preferably one to three modes, particularly preferably three modes, of the ventilation controller. In all three modes, RR and PIP are preferably adjusted in steps of 2/min or 1 mbar and the upper and lower limits for RR and PIP are preferably specified by the user. In the first mode “Basic”, the user additionally sets a desired upper value for the PIP, PIPprio. In the first mode, the controller prioritizes RR in order to achieve MV. This means that the setting range of RR is first exhausted before PIP is adjusted. An exception is the case when PIP is above PIPprio and the MV provided is to be reduced—in this case, PIP is first lowered to PIPprio before RR is adjusted. In a second mode, “Volume target”, the patient's compliance Crs measured by the ventilator is also used. The user can specify a target range for the tidal volume Vt here. Depending on the patient's compliance and the Positive End-Expiratory Pressure PEEP manually set by the medical staff, the ventilation controller adjusts RR and PIP so that the specified minute volume is prioritized with the desired Vt: MV=RR*(PIP−PEEP)=RR*Vt.

For comparison purposes, the ventilation controller can include a third, already known mode for minimizing the work of respiratory, which, for example, aims for an optimum respiratory rate for a given MV depending on Compliance Crs, Resistance R and Dead Space Vd.

The invention preferably comprises at least one of the following features: (i) use of PaCO2 as a control variable based on estimation of PaĈO2, (ii) use of the linear estimation function according to the above equation, wherein C1 and C2 can be adjusted during runtime, (iii) adaptive dead zone element with the above-mentioned setting options, (iv) ventilation controller mode “Basic”, (v) ventilation controller mode “Volume-Target”. Preferably, ventilation parameters from different functions are possible.

The invention preferably provides an inter-breath control. Further preferably, a total amount of ventilation is controlled, leaving a ventilation waveform to the ventilator.

The invention relates to a ventilator and a control device for controlling a respiratory gas source of a ventilator with a fan, in particular for newborns, wherein the control device comprises: (i) a target value providing unit which is configured to provide a target value of the arterial CO2 partial pressure, (ii) a minute volume determination unit which is configured to determine a target value of a minute volume based on the target value of the arterial CO2 partial pressure and a determined value or a value for the arterial CO2 partial pressure derived from the end-tidal CO2 partial pressure, if the determined value or the value derived from the end-tidal CO2 partial pressure for the arterial CO2 partial pressure lies outside a first predefined value range around the target value of the arterial CO2 partial pressure or lies within the first predefined value range for a time period less than a predefined time period, and (iii) a respiratory gas source control unit which is configured to receive the target value of the minute volume and to control the fan based on the target value of the minute volume. The ventilator according to the invention allows particularly gentle ventilation.

1. A ventilator (100, 600) for supplying respiratory gas, in particular to newborns, comprising
a respiratory gas source (110),
a control device (120, 610) for controlling the respiratory gas source (110),
a sensor device (130, 640) connected to the control device (120, 610) for detecting an end-tidal CO2 partial pressure (PetCO2),
an exchangeable respiratory gas hose (150) with at least a first connecting piece (151) for the respiratory gas hose (150) and a second connecting piece (152) for the respiratory gas hose (150) on a patient interface (140),
and a user interface (160) which is configured to receive user input,
wherein the control device (120, 610) comprises:
a target value providing unit (121) which is configured to provide a target value of the arterial CO2 partial pressure (PaCO2),
a minute volume determination unit (122) which is configured to determine a target value of a minute volume based on the target value of the arterial CO2 partial pressure (PaCO2) and a determined value for the arterial CO2 partial pressure or a value for the arterial CO2 partial pressure (Pa\hat{ }CO2) derived from the end-tidal CO2 partial pressure (PetCO2) if the determined value for the arterial CO2 partial pressure or the value for the arterial CO2 partial pressure (Pa\hat{ }CO2) derived from the end-tidal CO2 partial pressure (PetCO2) lies outside a first predefined value range (2 w) around the target value of the arterial CO2 partial pressure (PaCO2) or lies within the first predefined value range (2 w) for a time period less than a predefined time period (T), and
a respiratory gas source control unit (123) which is configured to receive the target value of the minute volume and to control the respiratory gas source based on the target value of the minute volume.

2. The ventilator (100, 600) according to claim 1, wherein the respiratory gas source control unit (123) is further configured to receive a preset value for the minute volume and to control the respiratory gas source (110) based on the preset value of the minute volume if the determined arterial CO2 partial pressure or the value for the arterial CO2 partial pressure (Pa\hat{ }CO2) derived from the end-tidal CO2 partial pressure (PetCO2) lies within the first predefined value range (2 w) for the predefined time period (T) and in a second predefined value range (2 L) around the target value of the arterial CO2 partial pressure (PaCO2) after the predefined time period (T), wherein the second predefined value range (2 L) is greater than the first predefined value range (2 w).

3. The ventilator (100, 600) according to any one of claim 1 or 2, wherein the minute volume determination unit (122) is configured to determine the target value for the minute volume based on the target value of the arterial CO2 partial pressure (PaCO2) and a value (Pa\hat{ }CO2) derived from the end-tidal CO2 partial pressure (PetCO2), wherein the derived value (Pa\hat{ }CO2) preferably corresponds to a piecewise linear approximation based on the end-tidal CO2 partial pressure (PetCO2).

4. The ventilator (100, 600) according to claim 3, wherein the derived value (Pa\hat{ }CO2) is calculated by the following equation:
$$Pa \hat{} CO 2 = C 1 \times PetCO 2 + C 2 ,$$
where Pa\hat{ }CO2 is the derived value, PetCO2 is the end-tidal CO2 partial pressure, and C1 and C2 are predefined parameters.

5. The ventilator (100, 600) according to one of the preceding claims, wherein the respiratory gas source control unit (123) is configured to determine a first maximum inspiratory pressure (PIP) and a first respiratory rate (RR) for controlling the respiratory gas source (110) based on the determined target value of the minute volume, and to control the respiratory gas source (110) based on the first determined maximum inspiratory pressure (PIP) and the first determined respiratory rate (RR).

6. The ventilator (100, 600) according to claim 5, wherein the respiratory gas source control unit (123) is further configured to receive a measured minute volume, the first determined maximum inspiratory pressure (PIP) and/or the first determined respiratory rate (RR), to determine a second maximum inspiratory pressure (PIP) and a second respiratory rate (RR) based at least on the measured minute volume, the first determined maximum inspiratory pressure (PIP) and/or the first determined respiratory rate (RR), and to control the respiratory gas source (110) based on the second determined maximum inspiratory pressure (PIP) and the second determined respiratory rate (RR).

7. The ventilator (100, 600) according to any one of claim 5 or 6, wherein the respiratory gas source control unit (123) is further configured to obtain a PIP value range having a lower PIP limit value and an upper PIP limit value for the maximum inspiratory pressure (PIP) and an RR value range having a lower RR limit value and an upper RR limit value for the respiratory rate (RR), and to control the respiratory gas source (110) based on the determined maximum inspiratory pressure (PIP) and the determined respiratory rate (RR) if the determined maximum inspiratory pressure (PIP) lies within the PIP value range and the determined respiratory rate (RR) lies within the RR value range.

8. The ventilator (100, 600) according to any one of claims 5 to 7, wherein the respiratory gas source control unit (123) is further configured to receive patient data, in particular a compliance (Crs) and/or a resistance (R), to determine a patient-dependent maximum inspiratory pressure (PIP) and a patient-dependent respiratory rate (RR) based at least on the determined minute volume and the patient data, and to control the respiratory gas source (110) based on the patient-dependent maximum inspiratory pressure (PIP) and the patient-dependent respiratory rate (RR).

9. The ventilator (100, 600) according to any one of claims 5 to 8, wherein the respiratory gas source control unit (123) is further configured to receive a target tidal volume range Vt having a lower Vt target limit value and an upper Vt target limit value, to determine a tidal volume-dependent maximum inspiratory pressure (PIP) and a tidal volume-dependent respiratory rate (RR) based at least on the determined minute volume and the target tidal volume range Vt, and to control the respiratory gas source (110) based on the tidal volume-dependent maximum inspiratory pressure (PIP) and the tidal volume-dependent respiratory rate (RR).

10. The ventilator (100, 600) according to claim 9, wherein the respiratory gas source control unit (123) is further configured, if a measured tidal volume is not within the target tidal volume range Vt, to determine a second tidal volume-dependent maximum inspiratory pressure (PIP2) based at least on the determined minute volume and to control the respiratory gas source (110) based on the second tidal volume-dependent maximum inspiratory pressure (PIP2).

11. The ventilator (100, 600) according to any one of claims 5 to 10, wherein the control device (120, 610) has at least one predefined mode selectable by the user interface (160), wherein the respiratory gas source control unit (123) is configured to control the respiratory gas source (110) based on the target minute volume value using the predefined mode.

12. The ventilator (100, 600) according to claim 11, wherein, in a first mode, the respiratory gas source control unit (123) is further configured to obtain a PIP value range having a lower PIP limit value and an upper PIP limit value for the maximum inspiratory pressure (PIP) and an RR value range having a lower RR limit value and an upper RR limit value for the respiratory rate (RR), to obtain a predefined value for the maximum inspiratory pressure (PIPprio) and to control the respiratory gas source (110) based on the determined respiratory rate (RR) if the determined value for the maximum inspiratory pressure (PIP) is smaller than the predefined value for the maximum inspiratory pressure (PIPprio), wherein the determined respiratory rate (RR) lies within the RR value range.

13. The ventilator (100, 600) according to claim 12, wherein the respiratory gas source control unit (123) is further configured to control the respiratory gas source (110) based on the determined maximum inspiratory pressure (PIP) and the determined respiratory rate (RR) if the determined respiratory rate (RR) corresponds to an upper RR limit value of the RR value range.

14. The ventilator (100, 600) according to any one of claim 12 or 13, wherein the respiratory gas source control unit (123) is further configured to control the respiratory gas source (110) based on the determined maximum inspiratory pressure (PIP) and the determined respiratory rate (RR) if the determined value for the maximum inspiratory pressure (PIP) is greater than the predefined value for the maximum inspiratory pressure (PIPprio).

15. The ventilator (100, 600) according to claim 14, wherein the respiratory gas source control unit (123) is further configured, if the determined maximum inspiratory pressure (PIP) corresponds to the predefined value for the maximum inspiratory pressure (PIPprio), to control the respiratory gas source (110) based on the determined respiratory rate (RR), wherein the determined respiratory rate (RR) lies within the RR value range, and, if the determined respiratory rate (RR) corresponds to a lower RR limit value of the RR value range, to control the respiratory gas source (110) based on the determined maximum inspiratory pressure (PIP).

16. The ventilator (100, 600) according to any one of claims 11 to 15, wherein, in a second mode, the respiratory gas source control unit (123) is further configured to receive a PIP value range having a lower PIP limit value and an upper PIP limit value for the maximum inspiratory pressure (PIP), an RR value range with a lower RR limit value and an upper RR limit value for the respiratory rate (RR) and a target tidal volume range (Vt) with a lower Vt target limit value and an upper Vt target limit value, based at least on the PIP value range, the RR value range and the target tidal volume range (Vt), to determine a maximum inspiratory pressure (PIP), and to control the respiratory gas source (110) based on the determined maximum inspiratory pressure (PIP) if a determined value for the tidal volume is not within the target tidal volume range (Vt), to determine a respiratory rate (RR) based at least on the PIP value range, the RR value range and the target tidal volume range (Vt), and to control the respiratory gas source (110) based on the determined respiratory rate (RR) if the determined value for the tidal volume lies within the target tidal volume range (Vt).

17. The ventilator (100, 600) according to claim 16, wherein the respiratory gas source control unit (123) is further configured to control the respiratory gas source (110) based on the determined maximum inspiratory pressure (PIP) and the determined respiratory rate (RR) if the determined value for the tidal volume is not within the target tidal volume range (Vt) and the maximum inspiratory pressure (PIP) corresponds to a lower or upper PIP limit value of the PIP value range.

18. Control device (120, 610) for controlling a respiratory gas source of a ventilator (100, 600), in particular for newborns, wherein the control device (120, 610) comprises:
a target value providing unit (121) which is configured to provide a target value of the arterial CO2 partial pressure (PaCO2),
a minute volume determination unit (122) which is configured to determine a target value of a minute volume based on the target value of the arterial CO2 partial pressure (PaCO2) and a determined value for the arterial CO2 partial pressure or a value for the arterial CO2 partial pressure (Pa\hat{ }CO2) derived from the end-tidal CO2 partial pressure (PetCO2) if the determined value for the arterial CO2 partial pressure or the value for the arterial CO2 partial pressure (Pa\hat{ }CO2) derived from the end-tidal CO2 partial pressure (PetCO2) lies outside a first predefined value range (2 w) around the target value of the arterial CO2 partial pressure (PaCO2) or lies within the first predefined value range (2 w) for a time period less than a predefined time period (T), and
a respiratory gas source control unit (123) which is configured to receive the target value of the minute volume and to control the respiratory gas source (110) based on the target value of the minute volume.

19. A method (700, 990) for controlling a ventilator, preferably a ventilator according to any one of claims 1 to 17, comprising:
Detecting (991) an end-tidal CO2 partial pressure (PetCO2),
Providing (992) a target value of the arterial CO2 partial pressure (PaCO2),
Determining (993), based on the target value of the arterial CO2 partial pressure (PaCO2) and a determined value for the arterial CO2 partial pressure or a value for the arterial CO2 partial pressure (Pa\hat{ }CO2) derived from the end-tidal CO2 partial pressure (PetCO2), a target value of a minute volume if the determined value for the arterial CO2 partial pressure or the value for the arterial CO2 partial pressure (Pa\hat{ }CO2) derived from the end-tidal CO2 partial pressure (PetCO2) lies outside a first predefined value range (2 w) around the target value of the arterial CO2 partial pressure (PaCO2) or lies within the first predefined value range (2 w) for a time period less than a predefined time period (T), and
Receiving (994) the target value of the minute volume and
Controlling (995) the respiratory gas source (110) based on the target value of the minute volume.

20. A computer program comprising program means for causing a control device (120, 610) according to claim 18 to perform the steps of the method (700, 990) according to claim 19 when the computer program is executed on the control device (120, 610).
